(19) United States
(12) Patent Application Publication
(10) Pub. No.: US 2018/0177430 A1
(43) Pub. Date: Jun. 28, 2018

(54) IMPEDANCE METHODS AND APPARATUSES USING ARRAYS OF BIPOLAR ELECTRODES

(71) Applicant: Impedimed Limited, Pinkenba, Queensland (AU)

(72) Inventors: Alfonso L. De Limon, Encinitas, CA (US); Udayan Rajendra Kanade, Kothrud (IN)

(21) Appl. No.: 15/739,620

(22) PCT Filed: May 20, 2016
(86) PCT No.: PCT/AU2016/050386

§ 371 Date: Dec. 22, 2017

Related U.S. Application Data
(60) Provisional application No. 62/185,116, filed on Jun. 26, 2015.

(51) Int. Cl. A61B 5/053; A61B 5/00

(52) U.S. Cl. CPC A61B 5/0537; A61B 5/6833; A61B 2562/043; A61B 5/0536; A61B 5/6831

(57) ABSTRACT

Apparatuses and methods for analyzing a region of a body (including a human body) by electrical impedance, using electrodes driven for bipolar stimulation (bipolar electrodes) and determining a frequency response in electrical properties at a plurality of sub-regions beneath an array of the bipolar electrodes that has been placed on a surface of the body. In particular, the methods and apparatuses described herein may be used to determine tissue wetness based on the change across frequencies based on bio-impedance measured with an array of bipolar electrodes.

### CROSS REFERENCE TO RELATED APPLICATIONS

This material may related to the following patents and patent applications, herein incorporated by reference in their entirety: U.S. patent application Ser. No. 13/715,788, filed on Dec. 14, 2012 (titled “METHODS FOR DETERMINING THE RELATIVE SPATIAL CHANGE IN SUBSURFACE RESISTIVITIES ACROSS FREQUENCIES IN TISSUE”); U.S. patent application Ser. No. 14/171,499, filed Feb. 3, 2014 (titled “DEVICES FOR DETERMINING THE RELATIVE SPATIAL CHANGE IN SUBSURFACE RESISTIVITIES ACROSS FREQUENCIES IN TISSUE”); and U.S. Pat. No. 8,068,906, issued Nov. 29, 2011 (titled “CARDIAC MONITORING SYSTEM”).

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Apparatuses, including devices and systems, as well as methods for determining impedance (including bio-impedance) using an array of end-to-end electrode pairs configured to operate as bipolar electrodes are described herein. For example, described herein are non-invasive methods and apparatuses for determining lung wetness using a sensor (e.g., adhesive patch sensor) including an array of bipolar electrodes.

### BACKGROUND

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

It has long been believed in the art that bipolar electrodes are inappropriate for measuring bio-impedance, and particularly for measuring bio-impedance to determine properties of a volume of tissue (e.g., beneath the skin), using impedance spectroscopy. Instead, tetrapolar arrays of electrodes have been used. In the bipolar method there are two electrodes which both apply and receive energy; near the bipolar electrodes the current density is higher than in other parts of the tissue, which results in a non-uniform impact into the total impedance measurement. The total impedance signal is a superposition of two components: the skin-electrode impedance (modified by blood flow-induced movement) and the original signal (e.g. caused by the blood flow). In practice it is difficult or impossible to separate them. See, e.g., Ambulatory Impedance Cardiography, The Systems and their Applications, G. Cybulski (Springer, 2011), Chapter 2, “Impedance Cardiography”, page 11.).

According to the prior art, bipolar electrodes are inappropriate because it is believed that contact impedances cannot be eliminated using a simple two electrode configuration; instead, tetrapolar electrodes (e.g., Tetrapolar Impedance Method) are used. See, e.g., K. S. Rabbani and M. A. Kadir, Bangladesh Journal of Medical Physics, Vol. 4, No.1, 2011, 67-74:67). The electrode impedance, for bipolar electrodes, is high, and makes the measurement of tissue impedance difficult.

Specifically, the prior art teaches away from the use of bipolar electrodes for impedance mapping of sub-surface tissue regions (e.g., regions beneath the skin), because the impedance of the skin and the electrode can be a problem for this kind of system due to unknown and varying contact impedance at each electrode site. The use of bipolar electrodes in such a system is believed to result in an indeterminate state, as there is only one measurement, while there are three variables in the system. See, e.g., Tissue Characterisation using an Impedance Spectroscopy Probe, Pedro Bertemes Filho, Doctoral Thesis, September 2002, Department of Medical Physics and Clinical Engineering, University of Sheffield, pages 8-11.

For example, when a two-electrode setup is used, the sensed voltage is measured not only across the unknown resistance, but also the resistance of the wires and contacts. In terms of bio-impedance measurement, not only the impedance of the body (Z_{body}) is measured but additionally the impedance of body, skin, wires and electrode impedances. In the following the combination of the skin, leads and electrode impedance that is believed to introduce errors in the measurement. See, e.g., p. 11-13, Development of a Capacitive Bioimpedance Measurement System, D. G. Abad, Thesis, Helmholtz-Institute for Biomedical Engineering (Rwth A-aachen University), August 2009.

Because of these concerns, bipolar (two-electrode) measurements are not considered by the prior art as suitable for bio-impedance measurement systems. Described herein are apparatuses (systems and devices) including arrays of bipolar electrodes for bipolar impedance mapping (e.g., measuring bio-impedance) of tissue beneath the skin. Although these systems and methods may be used in any bio-impedance mapping and/or imaging system, a particular application described in detail herein includes the use of bipolar electrodes and techniques to determine tissue fluid (e.g., water) content.

Tissue water content is an important and informative diagnostic parameter. Dehydration decreases cognitive and physical work capabilities, while the excessive hydration (swelling, edema) is a common symptom of cardiac, hepatic or renal pathology, malnutrition and many other pathologies and diseases. Edema causes muscle aches and pains and may affect the brain, causing headaches and irritability. Edema is a major symptom for deep venous thrombosis. It may be caused by allergies or more serious disorders of the kidney, bladder, heart, and liver, as well as food intolerance, poor diet (high sugar & salt intake), pregnancy, abuse of laxatives, diuretics, drugs, the use of contraceptive pills, hormone replacement therapy, phlebitis, etc.

For example, muscle water content (MWC) is a clinically useful measure of health. Monitoring of muscle water content can serve as an important indicator of body hydration status in athletes during the training as well as in soldiers during deployment. It is generally known that body hypohydration causes severe complications, health and performance problems, and that increasing body water weight loss causes increasing problems: water weight loss of up to 1% causes thirst, 2% may cause vague discomfort and oppression, 4% may cause increased effort for physical work, 5% may cause difficulty concentrating, 6% may cause impairment in exercise temperature regulation, increases in pulse and respiratory rate; 10% may cause spastic muscles; and 15% may cause death. Soldiers commonly dehydrate 2% -5% of body weight due to high rate of water loss from environmental exposure and performing stressful physical work. Dehydration by modest amounts (2%) decreases cognitive and physical work capabilities, while larger water losses have devastating effects on performance and health. Numerous pathologic signs and symptoms due to body dehydration include digestion problems, high blood pressure, muscle cramps, etc. MWC monitoring by an objective instrument may help prevent hazard thresholds. This is important because subjective indicators like thirst can be inadequate.

Control of MWC in athletes and soldiers could help in monitoring total body hydration during long-term endurance exercise or performance in hot weather conditions. In addition, tissue wetness may be particularly helpful in assessing lung wetness, which may be an important metric for treating cardiac disorders such as congestive heart failure.

Congestive heart failure (CHF) causes difficulty breathing because oxygen exchange in the lung is impeded by pulmonary congestion. The vast majority of CHF hospital admissions are because of difficulty breathing. Further, the high rate of CHF readmission (by some estimates approximately 24% within 30 days) is due to re-accumulation or inadequate removal of pulmonary congestion resulting in difficulty breathing. Currently, there is no quantifiable method or metric to identify pulmonary congestion and better prevent difficulty breathing and hospital admission. This problem is growing. In 2010, there was an estimated of 5.8 million CHF cases in the US, with over 670,000 new cases each year.

A subject suffering from CHF may be diagnosed using a physical exam and various imaging techniques to image the subject's chest. Treatment typically includes the use of vasodilators (e.g., ACEI/ARB), beta blockers, and diuretic therapy (e.g., Lasix). Management of treatment often proves difficult and unsuccessful. In particular, diuretic therapy is difficult for subjects and physicians to optimally manage. For example, changes in diet may require frequent changes in the diuretic therapy. Overuse (an underuse) of diuretic therapy may negatively impact clinical outcomes.

Pulmonary congestion is typically the result of high pulmonary blood pressures that drive fluid into the extravascular “spongy” interstitial lung tissue. High pulmonary blood pressures are present in subjects with elevated intravascular filling pressures as a result of heart failure. This high pulmonary blood pressure may also lead to increased amounts of fluid entering the extravascular space. Congestion within the extravascular interstitial lung tissue may prevent gas exchange ultimately, leading to a difficulty breathing that may require hospitalization. Hospital therapies are typically directed at reducing the pulmonary blood pressure by removing intravascular fluid with diuretic therapy. Although subject symptoms may improve, significant extravascular interstitial fluid may still be present. Subjects may feel well enough for discharge, but only a small change in pulmonary blood pressures will cause fluid to quickly re-accumulate, requiring readmission. Thus, subject symptoms do not reflect adequate treatment for the extent of the disease. Therefore, there is a need to detect and monitor extravascular interstitial fluid (e.g., lung wetness) and to provide an index or measure of the level extravascular interstitial fluid both instantaneously, and over time.

There are several methods for assessing total body water, as the most prominent indicator of hydration status, including methods based on bioelectrical impedance and conductance. For example, U.S. Pat. No. 4,008,712 to Nyboer discloses method and apparatus for performing electrical measurement of body electrical impedances to determine changes in total body water in normal and deranged states of the body, U.S. Pat. No. 5,615,689 to Kotler discloses a method of predicting body cell mass using impedance analysis, U.S. Pat. No. 6,280,396 to Clark discloses an apparatus and method for measuring subject's total body water content by measuring the impedance of the body, and U.S. Pat. No. 6,459,930 to Takehara et al. discloses a dehydration condition judging apparatus by measuring bioelectric impedance. However, these methods and systems have proven unreliable and difficult to implement. The aqueous tissues of the body, due to their dissolved electrolytes, are the major conductors of an electrical current, whereas body fat and bone have relatively poor conductance properties. Significant technical problems have hampered many such electrical methods for in vivo body composition analyses; impedance spectroscopy is an attempt to refine bio-impedance measurements, which measures resistance and reactance over a wide range of frequencies. A technique based on this approach is described in U.S. Pat. No. 6,125,297 to Siconolfi which describes a method and apparatus for determining volumes of body fluids in a subject using bioelectrical response spectroscopy.

Although various systems for using electrical energy have been proposed and developed, many of these systems are complex and difficult and expensive to implement. For example, systems such as electrical impedance imaging/tomography (EII/EIT) and applied potential tomography have been described elsewhere. For example, a system such as the one described in US 2007/0246046 to Teschner et al. (and others owned by the Draeger corporation) uses an electrical impedance tomography (EIT) method for reconstituting impedance distributions. In such systems, a plurality of electrodes may be arranged for this purpose on the conductive surface of the body being examined, and a control unit, usually a digital signal processor, typically ensures that a pair of (preferably) adjacent electrodes are each supplied consecutively with an electric alternating current (for example, 5 mA at 50 kHz), and the electric voltages are detected at the remaining electrodes acting as measuring electrodes and are sent to the control unit. Typically, a ring-shaped, equidistant arrangement of 16 electrodes is used, and these electrodes can be placed around the body of a subject, for example, with a belt. Alternating currents may be fed into two adjacent electrodes each, and the voltages are measured between the remaining current less electrode pairs acting as measuring electrodes and recorded by the control unit.

Other described EIT systems, such as those illustrated in U.S. Pat. No. 7,660,617, US 2010/0228143, and WO 91/019454, do not show evidence that measurements would not vary with subject habitus, e.g., body shape or geometry.

Unfortunately, electrical impedance methods have proven difficult to reliably and accurately implement for determining tissue wetness, and particularly lung wetness. Often, additional anthropometric terms (i.e., weight, age, gender, race, shoulder width, girth, waist-to-hip ratio, and body mass index) must be included in these previous prediction models to reduce the error of the estimate within acceptable boundaries. In addition, the reliability and reproducibility of the wetness estimates may vary depending on the geometry and placement of the electrodes. Thus, current methods and systems for assessing water content based on the bio-impedance of tissues may result in low accuracy, significant dependence of testing results on the anthropometrical features of the subject and on electrolyte balance.

There is therefore a need for a simple and highly accurate method and device for monitoring tissue hydration status that can be used in a broad range of field conditions.

### SUMMARY OF THE DISCLOSURE

Described herein are method and apparatuses (devices and systems) for determining impedance of a region of a body beneath an array of end-to-end electrodes configured to operate as bipolar electrode pairs. In particular, described herein are methods and apparatuses for determining changes in electrical properties based on the impedance across frequencies using arrays of bipolar electrodes. Changes in electrical properties (including or related to impedance) between different frequencies may be used to determine, estimate, or approximate properties of the body using an array of end-to-end (bipolar) electrodes placed on the body surface, for sub-regions located in the body beneath the electrode array.

For example, described herein are method and apparatuses (devices and systems) for determining bio-impedance of tissue using an array of end-to-end bipolar electrodes, and in particular, described herein are methods and apparatuses for determining changes in electrical properties based on the bio-impedance across frequencies using bipolar electrode arrays. Changes in electrical properties (including or related to bio-impedance) between different frequencies may be used to determine, estimate, or approximate tissue wetness, and particularly lung wetness using an array of end-to-end bipolar electrodes placed on the skin, for sub-regions located in the tissue beneath the electrode array. The arrays of electrodes described herein may be configured as a patch (e.g., adhesive patch) sensor having a plurality of bipolar electrode pairs (e.g., greater than 4 bipolar electrodes) on a substrate.

In general, the methods and apparatuses described herein may be used for detection, imaging and sensing apparatuses and methods in which arrays of electrodes (multi-electrode arrays) are used, particularly those that use electrical impedance. A non-limiting list of example include bio-impedance imaging, detection, monitoring and sensing, such as tumor (e.g., breast tumor, skin tumor) detection, etc., biological monitoring (e.g., lung/ventilation monitoring), cardiac detection (e.g., stroke detection), geophysical impedance testing, detection, imaging and monitoring (e.g., archeological detection via geophysical arrays), microfluidics applications (including electrophoresis, dielectrophoresis, electrorotation, polymerase chain reaction (PCR), surface micro fluidics, etc.), neurostimulation electrode array impedance measurement, and the like. Examples of such applications, including methods and apparatuses for performing them, are described in greater detail below.

In one broad the present invention seeks to provide a method of determining electrical properties of a region of a subject's body using bio-impedance of a tissue region, the method including:
attaching a sensor including a plurality of pairs of bipolar electrodes to a skin surface of the subject's body;
applying drive currents at a plurality of different frequencies to bipolar electrode pairs of the plurality of pairs of bipolar electrodes and measuring voltages at the bipolar electrode pairs; and
determining an estimate of electrical properties across at least two of the plurality of different frequencies for a plurality of regions beneath the sensor using the applied drive currents and measured voltages from the plurality of bipolar electrode pairs.

Typically applying drive currents at the plurality of different frequencies to each of the bipolar electrode pairs includes applying drive currents at a first frequency and at a second frequency.

Typically attaching the sensor includes attaching a sensor having N electrodes, wherein N is greater than 4.

Typically attaching the sensor includes attaching a sensor having N electrodes, wherein N is greater than 10.

Typically determining the estimate includes determining the estimate of electrical properties between a first frequency and a second frequency of the plurality of different frequencies for a plurality of regions beneath the sensor using the applied drive currents and measured voltages from the plurality of bipolar electrode pairs.

Typically determining the estimate includes determining an estimate of tissue wetness for at least some of the regions of the plurality of regions beneath the sensor.

Typically the method includes generating an indicator indicative of the estimate of tissue wetness.

Typically the method includes using a patch sensor including:
a substrate; and,
a plurality of pairs of bipolar electrodes on the substrate, wherein the substrate maintains a predetermined spacing between the electrodes.

Typically the method includes using an acquisition module to apply drive currents and determine the estimate of electrical properties.

Typically the method includes generating an indicator indicative of tissue wetness using the using an acquisition module to apply drive currents and determine the estimate of electrical properties.

In one broad the present invention seeks to provide apparatus for determining electrical properties of a region of a subject's body using bio-impedance of a tissue region, the apparatus including:
a sensor including a plurality of pairs of bipolar electrodes, the sensor being attached to a skin surface of the subject's body in use;
an acquisition module that:
   applies drive currents at a plurality of different frequencies to bipolar electrode pairs of the plurality of pairs of bipolar electrodes and measuring voltages at the bipolar electrode pairs; and
   determines an estimate of electrical properties across at least two of the plurality of different frequencies for a plurality of regions beneath the sensor using the applied drive currents and measured voltages from the plurality of bipolar electrode pairs.

Typically the sensor is a patch sensor including:
a substrate; and,
a plurality of pairs of bipolar electrodes on the substrate, wherein the substrate maintains a predetermined spacing between the electrodes.

Typically the patch sensor includes at least one substrate modification to enhance local flexibility of the substrate so that the patch sensor may conform to a contour of a subject's body,

Typically the substrate modifications to enhance local flexibility of the substrate include at least one of:
cut-out regions through the substrate;
slits cut through the substrate; and,
regions of material within the substrate having a greater flexibility than the substrate.

Typically the substrate is flexible and relatively inelastic, so that the spacing between each of the electrodes remains relatively fixed as the sensor is manipulated.

Typically the patch sensor further includes an adhesive hydrogel.

Typically the substrate at least one of:
is less than about 5 mils (0.127 mm) thick;
is a polyester material;
is a polyester material and an anti-bacterial titanium oxide material; and,
has a width of between about 0.5 inches (1.3 cm) and about 2.5 inches (6.4 cm).

Typically the plurality of electrodes include at least one of:
a rectangular shape on the substrate;
silver/silver chloride electrodes;
more than 6 elongate electrodes;
more than 10 electrodes; and,
more than 25 electrodes.

Typically the acquisition module includes:
an electrode drive unit configured to drive multiple different pairs of electrodes with at least two frequencies; and,
an electrode recording module that allows the acquisition module to record energy from the subject's skin in response to the applied energy between bipolar pairs of electrodes.

Typically the apparatus includes a data analysis unit that:
receives data from the acquisition unit indicative of the measured voltages and applied drive current;
uses the data to determine the estimate of the electrical properties.

In one broad the present invention seeks to provide a method of determining electrical properties of a region of a body beneath a sensor using impedance of the region, the method including:
attaching the sensor, wherein the sensor includes a plurality of pairs of bipolar electrodes to a surface of the body;
applying drive currents at a plurality of different frequencies to bipolar electrode pairs of the plurality of pairs of bipolar electrodes and measuring voltages at the bipolar electrode pairs; and
determining an estimate of electrical properties across at least two of the plurality of different frequencies for a plurality of regions beneath the sensor using the applied drive currents and measured voltages from the plurality of bipolar electrode pairs.

Typically the body is a body of a biological subject and the method includes determine electrical properties using bio-impedance of a tissue region by attaching the sensor to a skin surface of the subject's body.

In one broad the present invention seeks to provide a method of determining tissue wetness using bio-impedance of a tissue region, the method including:
attaching a sensor including a plurality of pairs of bipolar electrodes to a skin surface of a subject's body;
applying drive currents at a plurality of different frequencies to bipolar electrode pairs of the plurality of pairs of bipolar electrodes, and measuring voltages at the bipolar electrode pairs;
determining an estimate of tissue wetness for a plurality of regions beneath the sensor using the applied drive currents and measured voltages from the plurality of bipolar electrode pairs to determine a frequency response in electrical properties between at least two different frequencies.

In one broad the present invention seeks to provide apparatus for determining electrical properties of a region of a body beneath a sensor using impedance of the region, the apparatus including:
a sensor including a plurality of pairs of bipolar attached to a surface of the body;
an acquisition module that:
   applies drive currents at a plurality of different frequencies to bipolar electrode pairs of the plurality of pairs of bipolar electrodes and measuring voltages at the bipolar electrode pairs; and
   determines an estimate of electrical properties across at least two of the plurality of different frequencies for a plurality of regions beneath the sensor using the applied drive currents and measured voltages from the plurality of bipolar electrode pairs.

In one broad the present invention seeks to provide apparatus for determining tissue wetness using bio-impedance of a tissue region, the apparatus including:
a sensor including a plurality of pairs of bipolar electrodes, the sensor being attached to a skin surface of the subject's body in use;
an acquisition module that:
   applies drive currents at a plurality of different frequencies to bipolar electrode pairs of the plurality of pairs of bipolar electrodes and measuring voltages at the bipolar electrode pairs; and
   determines an estimate of electrical properties across at least two of the plurality of different frequencies for a plurality of regions beneath the sensor using the applied drive currents and measured voltages from the plurality of bipolar electrode pairs.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows one variation of an apparatus for determining bio-impedance spectroscopy for determining properties of tissue at a depth beneath the skin onto which the sensor is applied. For example, the apparatus in FIG. 1 may be configured to detect tissue (e.g., lung) wetness.

FIG. 2 illustrates one variation of a patch sensor (“patch”) including an array of bipolar electrode pairs that may be used.

FIG. 3A shows comparisons between standard tetrapolar measurements and bipolar measurements as described herein. Surprisingly, previous work (using a saline tank, not shown and circuit phantoms tests) confirmed that bipolar measurements could reconstruct tetrapolar measurements. In FIG. 3A, testing on a human subject using a system such as the one illustrated in FIG. 1 above, also showed that bipolar measurements could be used instead of tetrapolar measurements in the systems described herein. In this example, a modified script was designed to minimize the time between the acquisition of the tetrapolar and bipolar measurements to avoid temporal artifacts such as breathing.

### DETAILED DESCRIPTION

In general, described herein are apparatuses (systems and devices) and methods for the determining impedance using an array of electrode pairs configured to operate as bipolar arrays (e.g., applying energy, e.g., current, and sensing between energy, e.g., voltage, between pairs of the electrodes). The bipolar measurement apparatuses and methods described herein may be particularly well adapted for use in mapping bio-impedance in a region of tissue beneath the skin. For example, these apparatuses and methods may be adapted for use in detecting tissue wetness (including lung wetness). As mentioned above, and discussed in greater detail below, these methods and apparatuses are not limited to measuring/detecting/monitoring of bio-impedance or determining tissue wetness, but may be used for a variety of impedance measurement/monitoring applications, particularly where an array including a large number of electrodes is present, as the bipolar configuration may offer unexpected advantages over other (e.g., tetrapolar, tripolar, etc.) configurations commonly used and believed to be necessary.

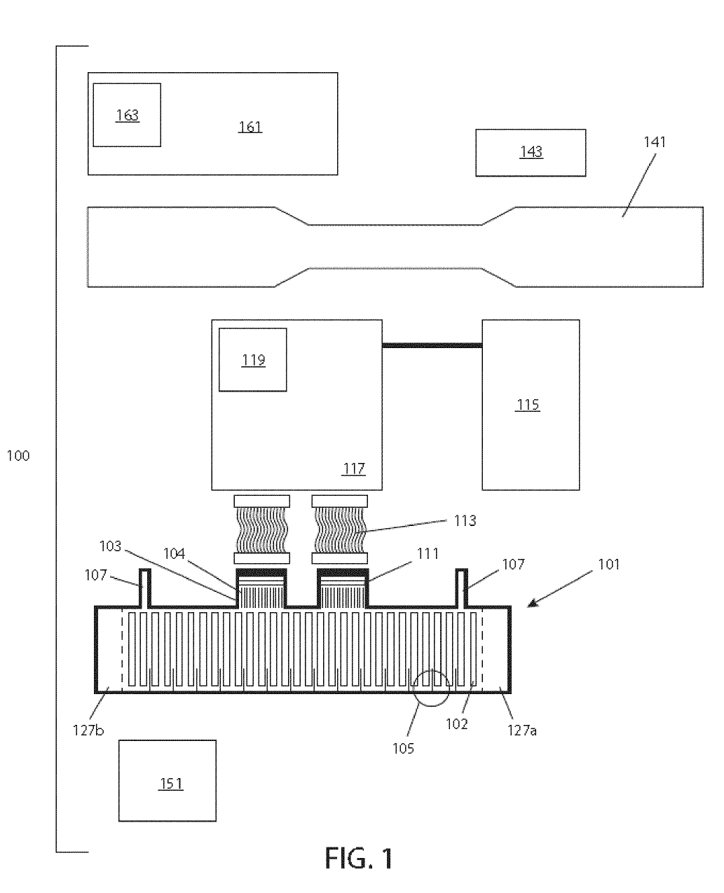

FIG. 1 illustrates one variation of an apparatus that is configured to determine lung wetness, and may use bipolar electrodes and bipolar tissue bio-impedance measurements. The apparatus in this example may measure electrical properties of biological tissue, such as conductivity or related and/or derived electrical properties, at multiple different frequencies (simultaneously or sequentially). The apparatus may then compare how these properties vary with frequency (e.g., frequency response) to determine “wetness”, for example, by determining how similar the change in electrical response with respect to frequency is compared to that of water. For example, the more similar the frequency response of a region of tissue to the frequency response of water (e.g., saline), the more likely that the region of tissue is “wet”. Thus, this system may examine electrical properties of tissue (such as conductivity or other, related or derived electrical properties) to assess tissue (e.g. lung) wetness.

This information can then be used to derive an indicator, indicative of the wetness. This could be in the form of an absolute wetness, or relative wetness, for example compared to a baseline or other reference wetness. The indicator could additionally or alternatively, be indicative of a medical condition associated with the wetness, such as a likelihood of the subject having a condition, or a degree of a condition.

In FIG. 1, the apparatus, which is shown configured as a system 100 including multiple, interacting and/or interconnecting parts, includes a patch sensor 101 (which may also be referred to as a patch or sensor patch, each having multiple individual electrodes, or an electrode array) that connects (via connecting cables 113) to an acquisition module 117 (AM), a power supply 115 (PS), and a data analysis unit 161 (DAU). Any of the systems described herein may also include connecting cables 113 connecting the patch sensor 101 to the acquisition module 117, a patient strap 141 that can be used to hold components of the system to the patient). The system may also include a diagnostic tool 151.

In general, many features of the patch 101 are similar to those described in US 2013/0165761 (application Ser. No. 13/715,788) and U.S. application Ser. No. 14/171,499, each herein incorporated by reference in its entirety. These patch electrodes may be adapted for use as bipolar electrode pairs. Typically any of the apparatuses, including patch electrodes, described herein may include at least four pairs of bipolar electrodes. In general, a bipolar electrode pair may be operated and configured to inject current between the two electrodes forming the pair, and measuring the resulting voltage between the same electrodes through which the current was injected (the bipolar pair). In the example apparatus (system) shown in FIG. 1, the patch 101, acquisition module (AM) 117 and data analysis unit 161 are adapted to deliver and receive bipolar stimulation from the array of possible electrodes 102 that may be operated as pairs of bipolar electrodes.

For example, the patch 101 may include a plurality of electrodes that are each configured for both injecting current (simulation electrodes) sensing voltage (sensing electrodes), and any two of them may be operated as a pair, to both (e.g., sequentially) apply current (or in some variations voltage) and to sense a resulting voltage (or in some variations, current) from which electrical properties (e.g., regional electrical properties) for one or more volumes of tissue beneath the patch may be determined. A patch 101 such as the one shown as an example in FIG. 1 may include at least four discrete electrode pairs (forming four bipolar electrode pairs, and thus having a minimum of four electrodes) positioned on a substrate. In this example the electrodes are a linear array of 1×31 electrodes that extend over an approximately 11 inch (28 cm) length of substrate. The electrodes 102 can be spaced apart from each other with a pitch of at least 0.100 inch (0.3 cm), such as a pitch of approximately 0.360 inch (0.9 cm). Alternatively, in some variations, the patch may include a two dimensional grid of electrodes that may form pairs in various combinations. The acquisition module is typically configured to control the electrodes to both deliver energy (e.g. current) and sense a response (e.g., voltage) from any or a predetermined sub-set of electrodes, and may communicate with the data analysis unit 161 to know when and what energy is applied and sensed between the individual bipolar pairs, so that this information may be used to determine the bio-electric properties of the region (e.g., sub-regions) beneath the patch.

The current electrodes (capable of forming bipolar pairs) shown in the example of FIG. 1 can be similar and/or dissimilar electrodes, so that bipolar pairs may include electrodes of the same or different types (e.g., different sizes and/or separations between the electrode pairs). For example, in some variations, some of the electrodes forming the bipolar pairs can have a smaller skin-contacting surface area than other electrodes in the pair, while in some variations the bipolar pairs are all of uniform size and/or shape. The electrodes are generally electrically conductive, and may be formed, for example of an electrically conducive metal, polymer, or the like, directly attached on a substrate.

In general, the substrate may be a flexible material that supports the electrodes, as well as adhesive, traces, connector(s), and other elements (including circuitry) on the patch. For example, the substrate may include a flexible material supporting electrodes, traces, connectors, etc. In some variations, the substrate is a polyester or other non-conductive, flexible material. The substrate may have any appropriate dimensions. The substrate may have any appropriate dimensions. For example, the substrate may be approximately 0.003 inch (0.01 cm) thick, and may be relatively long and wide (e.g., between about 0.8 inches (2 cm) and about 5 inches (13 cm) wide, between about 0.8 inches (2 cm) and about 3 inches (8 cm) wide, between about 4 inches (10 cm) and about 16 inches (40 cm) long, between about 4 inches (10 cm) and about 14 inches (35 cm) long, between about 5 inches (13 cm) and about 13 inches (33 cm) long, etc., greater than 0.8 inches (2 cm) wide, greater than 4 inches (10 cm) long, etc.).

The patch can be relatively large (e.g., greater than 4 inches (10 cm) long by 1 inch (2.5 cm) wide), and can allow each (or at least a majority) of the individual electrode contacts (e.g., voltage sensing pairs, and current injecting pairs) to make good electrical contact with the body (e.g., back) of a patient in order to take accurate, reliable and reproducible readings. However, it is also important that the spacing between individual electrodes in the array have a relatively fixed predetermined relationship relative to each other (e.g., the distance between the electrodes and between the sensing and driving electrode pairs). Although a rigid substrate would best preserve the predetermined spacing relationship between the electrodes, e.g., preventing buckling, bending, or the like, the more rigid the substrates are less likely to conform to the outer surface of the patient's body in a region where readings are to be taken. Thus, there is a tradeoff between how rigid (e.g., stiff) to make the substrate and how flexible (bendable) to make the substrate.

Accordingly, in one example, the patch includes a substrate and a plurality of electrodes on the substrate which are configured to form a plurality of pairs of current-injecting electrodes and a plurality of pairs of voltage detection electrodes, with the substrate maintaining a predetermined spacing between the electrodes. Additionally the patch includes at least one substrate modification to enhance local flexibility of the substrate so that the patch sensor may conform to a contour of a subject's body.

In this regard, this arrangement allows the patch to conform to the subject's body, thereby ensuring good electrical contact with the body, whilst substantially maintaining a physical spacing between the electrodes, which in turn allows for improved measurement accuracy.

In FIG. 1, the substrate of the patch includes a plurality of modified regions of the substrate that enhance the local flexibility of the substrate in these regions. For example, in FIG. 1, the patch 101 includes a plurality of flexible portions 105 that enhanced conformation of substrate/electrodes to a patient's back.

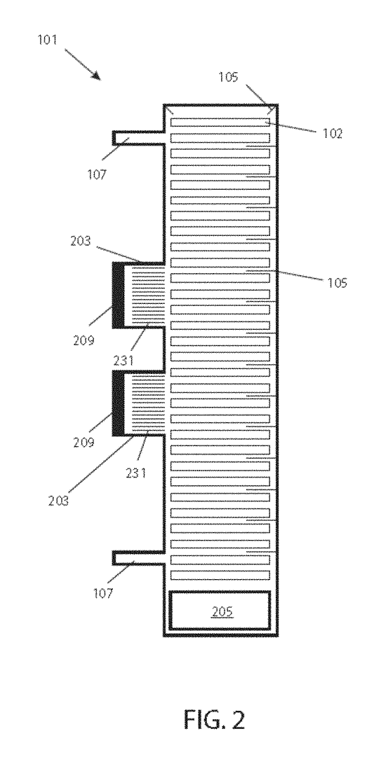

The flexible portions are shown as slits cut or formed into the substrate. In FIG. 1, the slits cut vertically from an outer elongate edge of the substrate between every other electrode 102. In FIG. 1, the slits are formed only on one side of the patch 101, for example, the side that is configured to be positioned opposite of the side of the patch that is positioned facing the spine (i.e. the side of patch 101 facing the bottom of the page as shown). FIG. 2, below, describes this in greater detail. However, it will be appreciated that alternative configurations could be used. For example, the slits could be provided on the side of the patch facing the spine, or could be provided on each side of the patch 101, depending on the preferred implementation. Additionally, the substrate modifications could be of alternative forms, such as openings, regions of different tensile elasticity or stiffness, regions of different materials, thickness or the like.

The system, and particularly the patch 101, shown in FIG. 1, can also include connecting tab portions 103. The connecting tabs 103 may be relatively stiff, such as to allow them to easily mate with connecting cables 113 or directly to the acquisition module 117 (or some other component, such as a wireless transmitter/receiver).

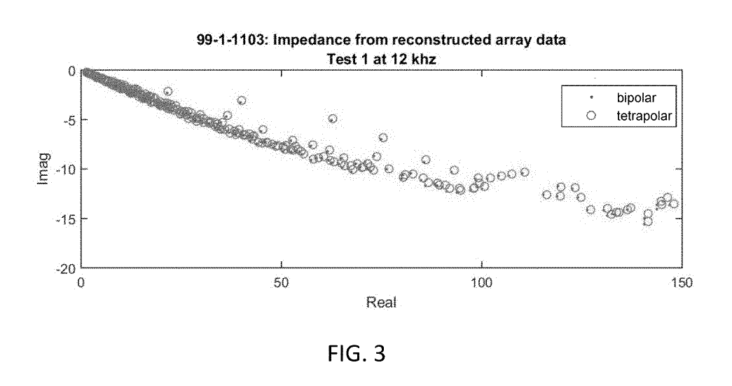

As mentioned, in FIG. 1 the flexible portions (substrate modification regions) are shown configured as slits although they may be configured generally to be regions of the substrate having an increased flexibility compared to an adjacent region. For example, in some variations the flexible portions/regions (or substrate modification regions) are cut-out regions in which shapes (e.g., circles, ovals, triangles, squares, diamonds, stars, etc.) are removed from the substrate and either allowed to be left open (see, e.g., FIG. 3), and can be filled or covered with an additional material having a greater flexibility than the rest of the substrate. In some variations the substrate may include stretchable regions.

In general, the individual electrodes 102 on the patch 101 may each have a surface area that is sized (e.g., is sufficiently large) to sufficiently reduce impedance encountered at electrode/patient interface. For example, electrodes 102 configured to inject current (stimulating electrodes) can comprise a skin-contacting surface large enough to avoid damage to skin and/or require high voltage drive signal. Alternatively or additionally, electrodes 102 configured for voltage or other signal sensing (sensing electrodes) can comprise a skin-contacting surface large enough to accurately record the desired signal, for example, as described briefly above, in some variations the sensor includes electrodes that are approximately 2 inches (5 cm) long, although they may be 1.5 inches (3.8 cm) long or smaller, and may be one or more order of magnitude narrower (e.g., less than about 0.2 inches (0.5 cm) wide). As mentioned, in general, the individual electrodes may be any appropriate conductive material, and may have a contact impedance of between about 10 Ohms-10 kOhms, such as between 10 Ohms-1000 Ohms. As mentioned above, in some variations, the stimulation electrodes and the sensing electrodes may have different surface areas. For example, the stimulation electrode surface area maybe greater than the sensing electrode surface area. For example the ratio of stimulation electrode surface area to sensing electrode surface area may be greater than 5:1, 10:1, 50:1; 100:1; 1000:1, etc. The contacting surface of the electrodes (e.g., the portion of the electrode that contacts the subject's skin) could have any appropriate shape, including a shape such as rectangular (e.g. square), elliptical (e.g. circular), polygonal, etc.

In general, any of these sensors (e.g., electrodes 102) could be configured as self-adhesive electrodes and may also include one or more agents to enhance electrical contact with the subject's skin. For example, the electrodes 102 may be hydrogel electrodes. In some variations the electrodes 102 include AG603 sensing gel with a thickness of about 0.025 inches (0.064 cm). In some variations, the volume resistivity of each electrode 102 is about 1000 ohm-cm maximum.

Any of the patch sensors 101 (patches) described herein may be adapted for connecting to a particular region of a patient's body, and in particular, a patient's back. Any of these patches may include one or more alignment elements, such as alignment tabs, to help align and couple the patch with a predetermined region of the subject's body.

Accordingly, in one example a non-invasive lung wetness patch sensor is provided that includes a substrate and a plurality of electrodes on the substrate configured to form a plurality of pairs of current-injecting electrodes and a plurality of pairs of voltage detection electrodes, with the substrate maintaining a predetermined spacing between the electrodes. A plurality of alignment tabs are provided extending from a lateral side of the substrate wherein the alignment tabs are between about 0.2 inches (0.5 cm) and about 2 inches (5 cm) long and greater than about 0.1 inches (0.3 cm) wide.

The use of alignment tabs allows the patch to be aligned relative to features of the subject's anatomy, such as the subject's spine. This can be used to assist in ensuring accurate and/or consistent placement of the patch on the subject. For example, this ensures the patch is positioned over the lung whose wetness is being measured, whilst ensuring that measurements are taken at the same location in the event that longitudinal monitoring is being performed.

In FIG. 1 and later figures, the patch 101 includes two alignment tabs 107 that may be used to position the array of electrodes 102 (forming bipolar electrode pairs) relative to patient anatomy. For example, when the system 100 is adapted to measure lung wetness, the patch 101 may be positioned in a location offset from the midline of the back (the spine), at a particular height relative to the shoulders. For example, the patch 101 may include superior and inferior alignment tabs that may help a user applying the patch 101 to the subject's back to align the electrodes 102 relative to the axis of the spine (e.g., lateral to medial positioning and/or superior to inferior positioning). For example, the patch 101 may be positioned using the alignment tabs 107 to place the left edge of electrode or geometric center of electrode relative to spine so that the medial (left) edge of electrodes is approximately 1.5 inches (4 cm) from center of spine. In FIG. 1, the alignment tabs 107 are approximately 1.5 inches (4 cm) long by 0.25 inches (0.6 cm) wide, and may include one or more alignment lines, arrows or other markers on the alignment tabs 107. Patch 101 can include one or more portions that are void of electrodes, adhesive and/or other additional material, such as superior grip portion 127a and inferior grip portion 127b shown in FIG. 1. Grip portions 127a and 127b can be grasped by a caregiver or other user during placement of patch 101 on the patient's back.

As mentioned above, the patch 101 may also include one or more connecting tabs. For example, a patch 101 may include connecting tabs 103 that include traces and a connector for connection to the acquisition module 117. The connecting tabs 103 may include a flex portion/region 104 that allows the connection to move slightly (e.g. allows the acquisition module to move relative to patch 101) without disturbing the patch 101 (e.g., moving it off of the subject's body). In addition, the connecting tabs 103 may include a stiffener 111 that assists in connection with the connecting cable(s) 113. The connecting tabs 103 may include insulated traces connecting to each electrode 102 in the patch 101. In FIG. 1, the connecting tabs 103 are each about 1.6 inches (4 cm) long by about 1.6 inches (4 cm) wide. In some variations, the patch 101 and attachment components are configured for placement of a patch 101 on the right side, or on the left side, and/or may be used on either the right side or the left side of a subject's back. For example, the patch may have at distinct “top” and “bottom” or the patch 101 may be used with either end acting as the top or bottom.

Although the patch 101 show in FIG. 1 and other examples is a unitary substrate with multiple individual electrodes, in some variations the patch may comprise multiple discrete substrates (or multiple discrete patches). These patches may be connected to each other or individually connected to an acquisition module.

As shown in FIG. 1, an acquisition module 117 may connect directly or indirectly (including wirelessly) to a patch 101, and generally coordinates the application of energy (e.g., current) at different frequencies, either concurrently or sequentially, from the drive energy between bipolar pairs of electrodes in the patch, and also coordinates the sensing of energy from the skin (e.g., sensing voltage) between the bipolar electrode pairs. The energy can be supplied in one or more modes, such as a constant-current mode. In some embodiments, the supplied energy is provided while maintaining a drive voltage less than 15V, such as less than 12V, less than 10V or less than 8V. In some embodiments, the energy is supplied while maintaining the injected current at a level between a lower threshold and a higher threshold, with or without maintaining the driving voltage as described above. In general, the acquisition module 117 may include a controller, configured as an electrode drive unit (e.g., electrode drive circuitry). The electrode drive circuitry may drive multiple, different pairs of electrodes with at least two frequencies. For example, the electrode drive circuitry/unit may drive at least 2 pairs of electrodes, at least 3-16 pairs of electrodes, etc. with at least 2 drive frequencies (e.g., such as at least two or more of approximately 8 kHz, 12 kHz, 20 kHz, 50 kHz, 100 kHz and 200 kHz). The drive frequencies may be, for example, divisive submultiples of a system clock. The clock may form part of the controller forming the acquisition module. For example the drive frequencies may be divisive submultiples of a clock frequency of approximately 39 MHz. In some variations, as described in US 2013/0165761, incorporated by reference above, the system (e.g., the acquisition module) operates at a lower and an upper drive frequency. For example, a lower frequency of approximately 8 kHz, 12 kHz, 20 kHz, or 50 kHz, and a higher frequency of approximately 20 kHz, 50 kHz, 100 kHz, 200 kHz, etc. As mentioned above, the energy applied can be constant current drive, constant voltage drive, or other signal that drives current from a first electrode of a bipolar pair to a second electrode of the bipolar pair, through the patient. For example, an acquisition module may be configured to include a constant current source driving at between 1 mA and 10 mA, such as a current of approximately 1 mA. The apparatus may be “voltage limited”, also as described above, to avoid harm to the patient (and may include safety features to prevent overdriving. The current source may be powered by a +/−12V power supply.

In general, the applied current may be a constant current source. In some variations, the drive signal may be multiple sinusoids delivered sequentially and/or simultaneously by the patch. For example, the acquisition module 117 may be configured to deliver 2-5 simultaneously delivered different frequency sinusoids. In some variations, the apparatus may be adapted to include a common ground, e.g. a large electrode placed on patient. This may allow “monopolar” stimulation and/or “monopolar” sensing from a single electrode 102 in the patch 101. In FIG. 1, as discussed above, the patch 101 and acquisition module 117 are adapted to operate in a bipolar configuration.

The acquisition module 117 may also include a user interface 119, such as one or more of a display (including a display, touchscreen, etc.), light such as an LED, audible transducer, tactile transducer, and combinations thereof. The acquisition module may also include a control (e.g., knob, button, dial, etc.). For example, the user interface 119 may be a graphical user interface (GUI). The user interface for the acquisition module 117 may display information about the status of the acquisition module 117 or other component of system 100, and may include one or more controls for controlling activity of the acquisition module 117 or other component of system 100 (e.g., start/stop, pause/resume, inputs for user information such as height, weight, age, gender, etc.).

In general, the acquisition module 117 (which, when adapted for use with the bipolar electrode arrays described herein, may be referred to as a bipolar acquisition module) typically includes an electrode recording module (e.g., electrode recording circuitry) that allows the acquisition module 117 to record energy from the subject's skin in response to the applied energy between bipolar pairs. For example, the acquisition module 117 may record voltages from a bipolar pairs of the electrodes 102, in response to the applied energy (e.g., current) between the same two electrode pairs. The data analysis unit is configured to receive data from the acquisition unit indicative of the measured voltages and applied drive current, using this to determine an estimate of the electrical properties across at least two of the plurality of different frequencies for a plurality of regions beneath the sensor. From this the data analysis unit determines an estimate of tissue wetness for at least some of the regions of the plurality of regions beneath the sensor, and optionally generates an indicator indicative of the tissue wetness. In this regard, the indicator can be in the form of a numerical value, graphical representation or the like.

Both the acquisition module (AM) 117 and data analysis unit 161 may be synchronized, so that the timing of applied energy and recorded tissue response between the bipolar pairs may be coordinated. A large number of very closely-timed (e.g. synchronized) applications/recordings of energy/response (at two or more frequencies) from the array of bipolar pairs may be coordinated by the acquisition module (AM) 117 and data analysis unit 161 through the sensing patch of bipolar electrode pairs, so that accurate recordings may be made while minimizing the duration of the session. The total data collection time period may be rapid enough to prevent movement/change artifacts in the user, as will be described in greater detail below. For example, the acquisition module 117 may record voltages from one or more pairs of the electrodes 102, including at least 1 pair, 3 pairs, 5 pairs, 10 pairs, etc. of electrodes 102.

In general, the acquisition module 117 both receives sensed response from a bipolar pair of electrodes (e.g., sensed voltage or in some variations current) and applied energy (e.g., applied current or in some variations, voltage), including which pair of bipolar electrodes (of electrodes 102) were used. The acquisition module 117 may store, transmit, process (e.g., filter, amplify, etc.) this information, and/or it may pass it directly on to a data analysis unit 161, which may be connected to the acquisition module 117 (including within the same housing) or it may be remote from the acquisition module 117.

In addition, as mentioned above, the acquisition module 117 may include an interface (e.g., interface 119) that receives subject-specific information about and/or from the subject. For example, the acquisition module 117 may include one or more inputs (e.g., buttons such as: keyboard; mouse; touchscreen; and combinations of these), and/or may receive inputs from additional measuring tools such as the diagnostic tool 151, as shown in FIG. 1. In some variations, acquisition module 117 and/or another component of system 100 can receive and/or record information such as clinician or other operator ID, Patient ID or other patient information, time, date, location, etc.

In FIG. 1 the acquisition module 117 is coupled to the patch 101 through connecting cables and may be separate from the patch 101. In some variations, the acquisition module 117 and the patch 101 are connected to each other directly. For example, at least a portion of the acquisition module 117 may be positioned on the patch; this may allow a reduction in the number of connecting wires between the acquisition module and the patch. Thus, the patch may include on-board electronics.

As mentioned and described in greater detail below, the acquisition module 117 may be integrated partially or entirely with the data analysis unit 161.

In some variations, the acquisition module 117 may include an interface or connector to one or more additional modules/devices. For example, an acquisition module 117 may include a USB Port or other data acquisition port for attachment to an external device. As mentioned, in some variations, system 100 (including the acquisition module 117) may include a wireless communication module, for wireless data transfer.

In one example, the acquisition module and/or data analysis unit include an electronic processing device, such as a microprocessor, microchip processor, logic gate configuration, firmware optionally associated with implementing logic such as an FPGA (Field Programmable Gate Array), or any other electronic device, system or arrangement, that operates to control the current source and voltage sensor. This arrangement typically includes digital to analogue converters (DACs) for coupling the processing device to amplifier for generating the required drive currents, and voltage buffer circuits coupled via analogue to digital converters (ADCs) to the electronic processing device, for returning a voltage signal.

As shown in FIG. 1, in general the apparatuses described herein may include a power supply 115. The power supply 115 may be a battery or a line in (wall power) supply, or a combination of these. Power supply 115 may include capacitive power supplies, or self-generating (e.g., solar) power supplies. Power supply 115 may include a rechargeable battery or other power supply (e.g. capacitor). The power supply 115 may be integrated into the acquisition module 117 and/or the data analysis unit 161 and/or patch 101, and may include a power conditioner to condition the power for use in applying energy to the patient, including safety features, such as safety features that limit one or more of current delivered and/or voltage applied.

In general, the apparatuses described herein include a data analysis unit 161 that may receive and/or analyze the sensed electrical energy (e.g., voltage) evoked by the applied energy (e.g., current). The data analysis unit 161 typically receives information (data) from the acquisition module 117. For example, the data analysis unit 161 may upload or otherwise access information from the acquisition module 117. For example, recorded voltage data, applied drive signal data, error data and/or timing data may be received by the data analysis unit 161 from the acquisition module 117. Additionally and/or alternatively, the acquisition module could perform at least some processing of the information, for example to calculate impedance values, such as magnitudes and/or phase angle values, with the impedance values being provided to the data analysis unit.

A data analysis unit 161 may include hardware, software, firmware, or the like that is configured to operate on the received bipolar data to estimate tissue wetness, e.g., lung wetness. For example, the data analysis unit 161 may be adapted to operate on the received data and perform a tissue wetness assessment based on voltages measured from the bipolar pairs of electrodes in response to single or multiple-frequency applied energy on the same bipolar electrodes. US 2013/0165761, previously incorporated by reference, describes and illustrates a variation of a method of determining/estimating tissue wetness based on multiple frequency information; although this example describes primarily tetrapolar electrodes (e.g., separate drive and sensing electrode pairs) the techniques and apparatuses described in this patent application may be adapted, as described herein, for use with bipolar pairs of electrodes. For example, the apparatuses described herein may determine regional electrical characteristics (such as conductivity/resistivity) for sub-regions of tissue beneath the patch at different frequencies to determine a frequency response for different regions beneath the patch. This frequency response may be compared to the frequency response for water (e.g., saline or other liquids that include water), and this comparison may be used to estimate tissue wetness. In some variations, the comparison of the frequency response may be made independently of body geometry. For example, the relative change in resistivities, which may look at the percent change in resistivities, dividing resistivity (e.g. a measured resistivity at a first location at a first frequency) by resistivity (e.g. a measured resistivity at the first location at a second, different frequency) resulting in a “unit less” measure (that may, in some variations, be independent of body geometry). Alternatively, in some variations the estimate of the frequency response may use body geometry or other patient diagnostic information to determine and/or compare the frequency response. For example, a correction factor based on body geometry may be used. Alternatively or additionally, body geometry may inform system 100 as to which portion of determined signal to use or the like. As discussed herein, body geometry may be entered manually or automatically, and may be determined in part from one or more tools, such as the diagnostic tools.

In general, the data analysis unit 161 may receive voltage and/or current information related to multiple frequency drive signal, along with the drive signals; drive signals may comprise sequential or simultaneous delivery of 2 or more frequencies. For example, for simultaneously driven signals, the recorded voltages can be split into frequency-correlated components (“bins”) and then analyzed by comparing magnitude/phase of the data in the various frequency “bins”. For example, a 256pt FFT with 1K bin widths that are centered at the two or more application frequencies may be used. The use of simultaneously driven frequencies may greatly reduce the time to apply/record over all of the electrode/electrode pairs used to calculate the signal and estimate wetness.

Any of the data analysis units 161 described herein may also include a user interface 163. For example, a data analysis unit 161 may include a user output component (e.g. screen) to “report” tissue wetness assessment. Alternatively, the output may be stored, and/or transmitted, e.g. including transmission back to the acquisition module 117 and/or to a separate component such as a third-party database (either with or without concurrent display).

In any of the variations described herein, the output may be an indicator of tissue (e.g., lung) wetness. For example, the apparatus may determine and present a quantitative assessment of lung wetness. The assessment may be a relative indicator, such as a numeric (e.g., 1-10) or qualitative assessment of lung wetness (e.g., dry, somewhat wet, wet, etc.). The assessment may be made for a partial portion of a lung, or an assessment of multiple discrete portions of a lung, or may be generalized to the entire lung, or for one lobe of the lung (or one side of the lung).

As mentioned above, the data analysis unit 161 may also include user interface (e.g., GUI) similar to the user interface described above for the acquisition module 117.

It will be appreciated from the above that the data analysis unit 161 could be of any appropriate form and could include a processing system, such as a suitably programmed PC, Internet terminal, lap-top, or hand-held PC, computer server, or the like. In one example the data analysis unit 161 is a tablet, smart phone, or other portable processing device, that is optionally connected to one or more computer servers, which could be distributed over a number of geographically separate locations, for example as part of a cloud based environment. In this example, the functionality provided by the data analysis unit could be distributed between multiple processing systems and/or devices, depending on the preferred implementation.

In variations including one or more connecting cables, as shown in FIG. 1, the connecting cables may be short. Alternatively, in some variations the apparatus may be configured so that the patch 101 is directly connected to the acquisition module 117, as mentioned above. Alternatively, the connecting cables may be integrated into the patch 101 and/or acquisition module 117.

Any of the apparatuses described herein may include one or more wearable holders that may be used to hold some of the components of the apparatus. For example, a patient strap 141 may be used, as shown in FIG. 1. In this variation, the strap may be worn over the subject's shoulder and may include connectors for some of the components. Alternatively or additionally, the wearable holding member (e.g., strap, belt, halter, etc.) may include a Velcro surface to which the components (e.g., acquisition module, battery, etc.) may attach. For example, in some variations, the strap 141 is configured to be positioned over the subject's shoulder when the patient is prone, and the acquisition module 117 may be attached to one side of the strap 141 while the battery (if separate from the acquisition module) may be positioned on the opposite side. In some variations the wearable holding member may be adapted for use with cradle 143.

In some variations the system does not include a strap. For example, the acquisition module, battery, etc. may be directly (e.g., adhesively) connected to the body, or may be placed near the subject's body, e.g., on a surface such as a bed, table, etc.

As mentioned above, any of the variations described herein may include a diagnostic tool. For example, a diagnostic tool may generally be a device to gather patient information. This patient information may be used by the systems (e.g., the data analysis unit 161) to assess tissue wetness. Examples of diagnostic tools include devices to gather back contour information, (e.g., mechanical or electromechanical measurement devices). Other diagnostic tools may include imaging devices, including devices for performing tissue imaging (e.g., MRI, X-Ray, Ultrasound Imager, etc.). In some variations the imaging device may include a camera. For example a camera may be used to take a picture of the subject and/or the setup for calculated estimation of “subject size/curvature”. In some variation the device may include software/firmware/hardware to assist the user in taking the image, so that the user could capture an optimal image. For example, the device may include a heads-up display input (e.g. live guide) to guide the user.

In some variations the apparatus may include control logic that, when executed on a processor causes the device to process the camera image to determine back curvature information. This information may be used to help position the patch and/or correct for patch position when calculating lung wetness. In some variations, the apparatus may include control logic to assist in taking an image (e.g., to guide to user to take an image by providing an orthogonally check, alignment (with patch) check, proper distance from the patient, etc.).

Any of the apparatuses described herein may also include one or more self-diagnostic and/or self-correcting capabilities. For example, U.S. Patent Application Publication No. 2013/0165761 (previously incorporated by reference in its entirety) described a system and method of determining which electrodes 102 to keep/reject when applying stimulation and/or recording signals for determining lung wetness. Such self-diagnostic capability can be incorporated into any of the elements of the apparatus, including the data analysis unit 161 and/or the acquisition module 117 and/or the patch 101.

Diagnostic capabilities may include: applicable patch tests, patch type testing, individual electrode testing (e.g. to determine one or more electrodes 102 “not to be used”, because of skin contact or breakage issues). For example, a voltage may be supplied between a bipolar electrode 102 pair (similar to normal operation), and the current measured. If the measured current is within expected range then the electrodes can be determined to be making good contact. If the measured current falls below expected range then it implies the impedance between electrodes is too high, thus poor or no contact. The test may be performed across different combination of pairs of electrodes 102 covering the whole patch. In some instances, a patch 101 with “bad” connections can be used (e.g., if below a maximum) by avoiding using those particular (i.e. identified as bad) electrodes 102 for forming bipolar pairs of electrodes for stimulating and/or sensing.

FIG. 2 illustrates another variation of a patch. In FIG. 2, the patch 101 includes at least a portion of an integrated acquisition module 205. The patch 101 may further include two alignment tabs 107 that may be used to position the array of electrodes relative to patient anatomy. The patch shown in FIG. 2 also includes flexing segments comprising slits 105 to enhance the substrate flexibility when worn on a contoured region of a subject's back, as described above. In addition to the slits in the substrate near the electrodes 102, the sensor patch may also include flexibility enhanced regions 231 (e.g., slits) in the connector tabs 203. Flexibility-enhancing regions (e.g., slits) can be positioned between any or all traces on a connecting tab and/or on the substrate between or otherwise proximate electrodes 102, e.g., between every trace, every 2nd trace, every 3rd trace, etc. If the flexibility enhancing region is a slit, the slit length may be any appropriate length, including the length of the connecting tab, minus clearance space for a connector 209, e.g. in the example shown in FIG. 2, at least 0.25″ (0.64 cm) clearance in an approximately 0.5″ (1.3 cm) long slit. As mentioned above, in this example, the slits are positioned along the lateral edge of the patch on one side (e.g., on the right side in FIG. 2, which would be positioned more laterally offset from the midline of the back on a patient. In FIG. 2, a slit is positioned after every second electrode, though a first slit is positioned between top two electrodes. Alternatively in some variations multiple slits are positioned no more than 2″ (5 cm) apart, e.g., approximately every 0.72″ (1.8 cm). Slits into the lateral side of the patch 101 may extend from (near or at) the lateral edge, and may extend as far as the midpoint (or less) of nearest electrodes. In FIG. 2, the slit has a length of approximately 0.5″ (1.3 cm), such as 0.484″ (1.23 cm). In some variations, the patch 101 includes a slit at each corner of the patch. FIG. 2 shows slits at the superior two corners, however slits could be positioned at any or all of the four corners.

### Bipolar Measurements

In general, the systems and apparatuses described herein (including the exemplary system above) may include an array of electrodes adapted to be used as bipolar electrode pairs, for example, for making end-to-end impedance measurements. As mentioned above, previous systems (including US 2013/0165761, discussed above) use tetrapolar arrays of electrodes. In contrast, as described below, the apparatuses and methods herein use bipolar pairs of electrodes to determine bio-impedance in the region beneath the device. As will be discussed in greater detail below, it can be shown that a minimum of four pairs of bipolar electrodes are required to achieve an equivalent set of measurements compared to a single tetrapolar set of electrodes (e.g., a pair of sensing electrodes and a separate set of driving electrodes). Although the greater number of bipolar pairs required for this simple comparison suggests that the number of bipolar pairs of electrodes would be prohibitively large, surprisingly both the theoretical analysis and the proof-of-principle empirical examples described herein show that there is instead both numerical and signal quality advantages that were not previously suggested in the art.

These advantages may allow the apparatuses described herein to make substantially fewer measurements, particularly as compared to tetrapolar systems, while still recovering an equivalent amount of information, which in some cases may be more robustly sensed, because of the use of the bipolar electrode pairs. More surprisingly, the multi-frequency methods described herein, in which bipolar impedance measurements are used to compare between different frequencies results provide robust and signals, despite the prior art belief that (as described above) the use of bipolar measurements for determining bio-impedance would result in signals that were contaminated (or overwhelmed by) skin impedance. In particular, the inventors have found (as described herein) that the skin impedance is not a significant source of noise, particularly when using bipolar (e.g., end-to-end) electrodes for comparison of bio-impedance across frequencies as performed by the methods and apparatuses described herein. These methods allow bipolar (end to end) stimulation and sensing from the same locations. As described the theoretical description, provided below (see, e.g., equation 34), the skin impedance that may otherwise be prohibitive, cancels out (e.g., when comparing across frequencies), as described herein. As a result, since the resulting skin impedance will effectively cancel out, the methods and apparatuses described herein may use an array of bipolar, rather than tetrapolar, electrodes.

In examples in which a one-dimensional array of electrodes (as illustrated above in FIGS. 1 and 2, e.g., showing an array of 31 electrodes), any two of the electrodes in the array may be used to form the bipolar pairs. For example, when using a 31 electrode array, there are greater than 200,000 possible tetra-polar measurements that can be performed, many of which may be redundant. Further, it may be difficult to determine what minimum number of adequate measurements (tetrapolar electrode measurements) are necessary in order to provide a sufficient and robust data set in order to use bio-impedance for determination of, for example, tissue wetness. As described herein, not only are end-to-end (e.g., bi-polar) measurement equivalent to tetra-polar arrays, but the signals recorded from bipolar electrodes, despite the teachings of the prior art, may actually provide substantial advantage in signal strength, recording speed, and reliability over otherwise equivalent tetrapolar readings.

For example, given N electrodes capable of measuring the voltage on the surface of a body, as modeled by equations 2-5, below. Modeling the divergence of the current in the body as zero, except at the electrodes, which can be reformulated in terms of equations 6-9, below, assuming Kirchhoff law (equation 10). Then, assuming +/− current point sources (as in equation 11), and that we measure only the voltage drop (as in equation 12), then the symmetries in the tetra-polar readings are apparent in equations 13-15 (note that equation 15 is reciprocity). Based on these symmetries, a set of models equations (e.g., equations 16-27) may be written in which two of them are related to the third via f̂(ab)+f̂(bc)=f̂(ac). Because these model parameters must have the same solution up to a constant, the relationship of equations 28-29 provides the special case of equation 30. Using these relationships, the equation (proof) as shown in equation 41 provides that any tetra-polar voltage (V̂A(ab)_(cd)) is equivalent to four bipolar (end to end) measurements in the configuration: V̂(ca)_(ca)−V̂(da)_(da)−V̂(cb)_(cb)+V̂(db)_(db).

Although this may appear to initially be a disadvantage (as it implies that four bipolar pairs of electrodes are needed to provide an equivalent measure to one set of tetrapolar electrodes), as the dimensions of the array of electrodes (bipolar vs. tetrapolar) increases, the space of tetra-polar arrays is roughly (N choose 2)̂2, while the space of bi-polar arrays is only N choose 2; using equation 41 (or equation 77), below, all tetra-polar measurements can be reconstructed given the bi-polar measurements. Moreover, the bi-polar arrays are complete, resulting in a redundancy of our 31 electrode array such that there are only 465 unique pairs (assuming no noise), in contrast to the greater than 200,000 unique tetrapolar electrode arrangements.

Thus, the use of bipolar electrodes, particularly with relatively large electrode arrays, reduces by a square-root, the number of measurements that have to be taken. This is particularly advantageous as N becomes larger, in particular when using a two dimensional (2D) patch. Moreover, as mentioned above, the signal size of bi-polar measurements is typically larger than those from tetra-polar arrays.

In addition, as the number of unique pairs (bipolar pairs) is smaller, requiring fewer measurements to be made, the measurement process may be much quicker than when using tetrapolar electrodes. For example, a one dimensional array of 31 electrodes (N=31) measured uniquely using bipolar electrodes may be performed hundreds of times faster, compared to equivalent tetrapolar measurements. This difference may become even more pronounced when using a 2D array, as a much smaller number of unique pairs may result in a much faster complete data set when using bipolar, rather than tetrapolar, measurements. In general, the speed of recording the bio-impedance data set (e.g., the complete or nearly-complete set of unique measurements) may also affect the quality of the data collected. Assuming that there are no external electromagnetic forces being applied, all of the measurements (e.g., all of the 465 bipolar measurements) should be performed sufficiently fast so that the first and last measurements are taken within a reasonable amount of time (e.g., less than 5 seconds, less than 4 seconds, less than 3 seconds, less than 2 seconds, less than 1 second, less than 0.9 seconds, less than 0.8 seconds, less than 0.7 seconds, less than 0.6 seconds, less than 0.5 seconds, less than 0.4 seconds, less than 0.3 seconds, less than 0.2 seconds, less than 0.1 second, etc.), such that the recording conditions are relatively unchanged (e.g., body position, respiratory cycle, etc. In general, the faster the bipolar measurements forming a complete set of the unique bipolar pairs are taken, the better, so that, all of the measurements are taken at effectively the same time (e.g., typically in less than 0.5 seconds). Although the system may be configured to tolerate some change in tissue/body position (e.g., due to respiration) it is preferable to minimize such noise, e.g., by speeding up the data acquisition.

As mentioned above, another advantage of bipolar measurements compared to tetrapolar measurements is the relative side of the resulting measurements. Bipolar measurements tend to be larger than tetrapolar, and have a more uniform distribution (e.g., all signals tend to be about the same size). Thus, signals can be accurate within a reasonable range of values recorded. In addition, there is less variation in signal strength across frequencies with bipolar versus tetrapolar measurements. Thus, even when measuring multi-tonal (e.g., across frequencies, such as at two frequencies), when taking bipolar measurements there is less of a need to increase the current at different frequencies in order to get equivalently sized measurements, unlike with tetrapolar measurements. In general, because of the advantage mentioned above (and potentially others), the hardware necessary (including electrodes, acquisition module 117, data analysis unit 161, etc.) may be simpler, particular compared to the tetrapolar measurement. For example bipolar systems (as opposed to tetrapolar systems) may require fewer measurements, and fewer signal processing (e.g. amplification, switching/increasing applied current, etc.). In addition, the use of multiple bipolar pairs as opposed to tetrapolar electrodes may permit the use of concurrent or simultaneous application of energy at different frequencies, to so you the bipolar electrode pairs are driven at multiple frequencies at the same time, rather than dividing up the application of driving energy at different frequencies. This may also simplify the power management requirements (e.g. allowing a smaller battery), smaller battery, etc.).

The theory of operation section below provides a mathematical description of the use of bipolar electrodes in the multi-tonal (multi-frequency)/across-frequency bio-impedance comparisons (e.g., to determine lung wetness or other biological conditions), and this model is supported by empirical evidence, as described in FIG. 3. This theory assumes that there are no external electromagnetics (electromagnetic fields) operating on the tissue at the time the bipolar measurements are made (and over the same range of values). Further, the model assumes that all of the measurements made are taken on the same electrical body (e.g., tissue region). These assumptions are reasonable for the applied currents and working conditions for the apparatuses and methods described herein.

In addition to the advantage mentioned above, the electrodes used to create the bipolar electrode pairs may be smaller (as less current may be used) compared to tetrapolar electrodes to get comparable data. In some variations, the electrodes (e.g., electrode array) or sensor pad used as described herein may be configured (e.g., without adhesive) as a momentary contact electrode. Thus, the electrodes in some variation may be used without a hydrogel, particularly when configured as a momentary contact electrode array. Such configurations are particularly useful for momentary contact electrodes (e.g., where contact time may be less than 0.5 seconds).

### Theory of Operation

As discussed above, the use of bipolar electrodes in a system and/or method that rapidly measures and compares impedance measurements across frequencies (e.g., using ratios across frequencies) so that the otherwise prohibitively large skin impedance may be cancelled out. For the purposes of illustrating the theory of operation when using bipolar electrodes in this manner, assume (as a non-binding example) a system in which there is a probe with:

N=31 (1)

individual electrodes, which may be divided as either bipolar electrode pairs or as tetrapolar electrodes.

Initially, let there be a body with Ω with boundary ∂Ω. The probes are subsets of the boundary {Eᵢ}ᵢ₌₁^{N} with each Eᵢ ∈ ∂Ω. The equations for the current probed human body are:

∇·(σ∇u)=0 in Ω (2)

\hat{n}·(σ∇u)=0 on ∂Ω\UᵢEᵢ (3)

u=vᵢ on Eᵢ (4)

∫_{E}_{ᵢ}σ(∇u)·d\hat{n}=fᵢ on Eᵢ (5)

Boundary value problems can be created from these equations in more than one way. For example, one way to create a boundary value problem is to treat the vᵢs as unknowns, along with the unknown field u. This creates the following boundary value problem:

∇·(σ∇u)=0 in Ω (6)

\hat{n}·(σ∇u)=0 on ∂Ω\UᵢEᵢ (7)

u−vᵢ=0 on Eᵢ (8)

∫_{E}_{ᵢ}σ(∇u)·d\hat{n}=fᵢ on Eᵢ (9)

The boundary value problem (6)-(9) can be solved uniquely up to a constant. Solving the boundary value problem (6)-(9) will give values of u as well as values of vᵢ. Equation (8) can be called an “equipotential boundary condition”, where we are specifying that the potential on each Eᵢ should be the same, regardless of what it is. Equation (8) falls one real number short of unique determination of the solution (per electrode), and this extra real number is provided (per electrode) by the equation (9). Equations (6)-(9) are solvable if and only if:

$${\sum }_{i = 1}^{N} {f}_{i} = 0 \qquad ( 10 )$$

The physics of the bio-impedance systems described herein (such as the system illustrated in FIG. 1) can be modeled accurately by Equations (6)-(9), with further restrictions. These systems cannot create any possible boundary condition {fᵢ}ᵢ, but only a certain kind of boundary conditions:

f=f_{(ab)}:=∂ₐ−∂_{b} (11)

Where ∂ᵢ stands for the Kronecker delta function. Furthermore, these systems cannot measure all the u's and v's solved in equations (6)-(9), but can only measure:

v_{(cd)}^{(ab)}:=v_{c}^{(ab)}−v_{d}^{(ab)} (12)

It is important to see that since solving the equations (6)-(9) solves for u and vᵢ determined uniquely up to a constant, the readings v₍ᵢⱼ₎ are uniquely determined (the constant from vᵢ and vⱼ cancels out). Because of the unique determination, the following laws are obvious:

v_{(cd)}^{(ab)}=−v_{(cd)}^{(ba)} (13)

v_{(cd)}^{(ab)}=−v_{(dc)}^{(ab)} (14)

v_{(cd)}^{(ab)}=v_{(ab)}^{(cd)} (15)

Suppose we have four electrodes, a, b, c, d. Suppose we have the three physics:

∇·(σ∇u^{(ab)})=0 in Ω (16)

\hat{n}·(σ∇u^{(ab)})=0 on ∂Ω\UᵢEᵢ (17)

u^{(ab)}−vᵢ^{(ab)}=0 on Eᵢ (18)

∫_{E}_{ᵢ}σ(∇u^{(ab)})·d\hat{n}=fᵢ^{(ab)} on Eᵢ (19)

∇·(σ∇u^{(bc)})=0 in Ω (20)

\hat{n}·(σ∇u^{(bc)})=0 on ∂Ω\UᵢEᵢ (21)

u^{(bc)}−vᵢ^{(bc)}=0 on Eᵢ (22)

∫_{E}_{ᵢ}σ(∇u^{(bc)})·d\hat{n}=fᵢ^{(bc)} on Eᵢ (23)

∇·(σ∇u^{(ac)})=0 in Ω (24)

\hat{n}·(σ∇u^{(ac)})=0 on ∂Ω\UᵢEᵢ (25)

u^{(ac)}−vᵢ^{(ac)}=0 on Eᵢ (26)

∫_{E}_{ᵢ}σ(∇u^{(ac)})·d\hat{n}=fᵢ^{(ac)} on Eᵢ (27)

Since f^{(ab)}+f^{(bc)}=f^{(ac)}, we see that (u^{(ab)}+u^{(bc)}, v^{(ab)}+v^{(bc)}) is as good a solution of (24-27) as is (u^{(ac)}, v^{(ac)}) These two solutions can only differ by up to a constant. Let us call this constant k^{abc}. We have the relations:

u^{(ab)}+u^{(bc)}=u^{(ac)}+k^{(abc)} (28)

v^{(ab)}+v^{(bc)}=v^{(ac)}+k^{(abc)} (29)

We can develop the following relations between these:

$$\begin{matrix} {v}^{( ab )} + {v}^{( ba )} = {k}^{( aba )} & ( 30 ) \\ {k}^{( bca )} - {k}^{( cad )} + {k}^{( adb )} - {k}^{( dbc )} = {v}^{( bc )} + {v}^{( ca )} - {v}^{( ba )} - ( {v}^{( ca )} + {v}^{( ad )} - {v}^{( cd )} ) + {v}^{( ad )} + {v}^{( db )} - {v}^{( ab )} - ( {v}^{( db )} + {v}^{( bc )} - {v}^{( dc )} ) & ( 31 ) \\ = - {v}^{( ba )} - {v}^{( ab )} + {v}^{( cd )} + {v}^{( dc )} & ( 32 ) \\ = - {k}^{( aba )} + {k}^{( cdc )} & ( 33 )\end{matrix}$$

A very interesting relation is:

$$\begin{matrix}{v}_{( ca )}^{( ca )} - {v}_{( da )}^{( da )} - {v}_{( cb )}^{( cb )} + {v}_{( db )}^{( db )} {=}_{( 13 )} {v}_{( ca )}^{( ca )} + {v}_{( da )}^{( ad )} + {v}_{( cb )}^{( bc )} + {v}_{( db )}^{( db )} & ( 34 ) \\ {=}_{( 12 )} {v}_{c}^{( ca )} + {v}_{d}^{( ad )} + {v}_{c}^{( bc )} + {v}_{d}^{( db )} - {v}_{a}^{( ca )} - {v}_{a}^{( ad )} - {v}_{b}^{( bc )} - {v}_{b}^{( db )} & ( 35 ) \\ {=}_{( 29 )} {v}_{c}^{( ba )} + {k}^{( bca )} - {v}_{a}^{( cd )} - {k}^{( cad )} + {v}_{d}^{( ab )} + {k}^{( adc )} - {v}_{b}^{( dc )} - {k}^{( dbc )} & ( 36 ) \\ {=}_{( 33 )} {v}_{c}^{( ba )} + {v}_{d}^{( ab )} - {v}_{a}^{( cd )} - {v}_{b}^{( dc )} - {k}^{( abc )} + {k}^{( cdc )} & ( 37 ) \\ {=}_{( 30 )} {v}_{c}^{( ba )} - {v}_{d}^{( ba )} + {v}_{a}^{( dc )} - {v}_{b}^{( dc )} & ( 38 ) \\ {=}_{( 12 )} {v}_{( cd )}^{( ba )} + {v}_{( ab )}^{( dc )} & ( 39 ) \\ {=}_{( 13 )} - {v}_{( cd )}^{( ab )} - {v}_{( ab )}^{( cd )} & ( 40 ) \\ {=}_{( 15 )} - 2 {v}_{( cd )}^{( ab )} & ( 41 )\end{matrix}$$

The use of bipolar electrodes and bipolar methods for determining changes in bio-impedance across frequencies described in the analysis above has been empirically confirmed. For example, an array of bipolar electrodes (similar to those shown above in FIG. 2), was used in a saline tank and tested (e.g., using circuit phantoms) to confirm that bipolar measurements could reconstruct tetrapolar measurements. Further, as described in FIG. 3A, a similar set of experiments was used on a human subject, showing that there is no significant difference in determining changes in bio-impedance across frequencies using either bipolar electrode arrays or tetrapolar electrode arrays, and corresponding methods. For example, in FIG. 3A a modified script was used to take both tetrapolar and bipolar measurements from an electrode array (patch array) such as the one shown in FIG. 2, applied to a subject's skin. The time between tetrapolar and bipolar measurements was minimized, so as to avoid temporal artifacts, e.g., due to breathing, body movement, etc.

As shown in FIG. 3A, the bio-impedance determined from reconstructed array data using either bipolar (shown as dots) and corresponding tetrapolar (shown as circles) electrode sets at 12 kHz shows that the two techniques are highly correlated. This data was generated by placing a healthy subject in a prone position on bed, after skin preparation (e.g., an abrasive scrub and alcoholic cleanse). A 31 electrode hydrogel patch was place on subject's back, one inch from spine with the top of the patch starting at T2. Several hundred Wenner-Schlumberger arrays we selected and decomposed into bipolar arrays. The two sets of arrays were taken back to back to minimize temporal artifacts such as breathing. The subject was asked to hold from breathing and a second test was performed in which the subject took normal breaths. There were no significant differences between both tests, thus the bi-polar arrays were taken sufficiently fast to avoid temporal effects. Comparing tetrapolar arrays to their bipolar counterparts (Eq. 41) also yielded no appreciable difference between both sets of measurements (FIG. 3A), showing experimentally that the map between bipolar and tetrapolar arrays work in a clinical settings.

The apparatuses and methods described above can be used not only to form a system for detection of lung wetness, as described above, but may generally be used with (or as part of) electromechanical systems, and particularly those that examine changes in bio-impedance across (or between) frequencies, including bio-impedance imaging systems and the like.

### Additional Examples

The general apparatuses (e.g., bi-polar arrays and analysis apparatuses) and method of using them described herein may be used in any application and as part of any apparatus (system or method) in which the electrical impedance may be measured and/or estimated, and particularly systems in which electrical impedance at multiple frequencies are measured and/or compared. Specific alternatives embodiments and applications are described herein.

In addition to lung wetness, other biological applications (e.g., bio-impedance techniques) may also benefit from the use of the bi-polar electrode arrays and techniques described herein. For example, breast tumor detection via bio-impedance. Electrical impedance tomography (EIT) is for imaging includes (as one example) electrical impedance computerized mammography, which has been used for breast diagnostics. In general, as described above, electrical impedance imaging works on the principle of tissues having different electrical properties (conductivities and resistance) which may depend on their cell structure and pathology. Breast cancers are known to differ significantly from those of normal breast tissues (e.g., the conductivity of cancerous tissue may be different from no-cancerous tissue), and these differences may be exploited by EIT as a modality of breast cancer imaging. An exemplary EIT system may include a hand-held scanning probe and a computer screen that displays two-dimensional images of the breast. In contrast to known and currently used electrodes, based on the work described herein, an array of bipolar electrodes may be placed on the patient's arm, and electric current transmitted through the bipolar array of electrodes into the body. The current travels through the tissue (e.g., breast) where the electrical conductivity may be sensed and measured by the system similar to that as described above; an image may then be generated from the measurements of electrical impedance and displayed.

Similarly, the methods described herein may be used to detect, sense and/or monitor other types of biological disorders, including other cancers, such as skin cancers. For example, electrical impedance spectroscopy (EIS) may be used to apply an electrical signal to the skin to detect skin cancer; the use of an array of bipolar electrodes as described herein may provide enhanced accuracy and specificity in detection.

Electrical Impedance Tomography (EIT) using the improvements described herein (e.g., bipolar arrays) may also be used for enhanced biological imaging techniques, for example by providing images of the internal impedance of a subject that can be rapidly collected with arrays of external bipolar electrodes positioned on or around the subject's body. For example, this may be fast, inexpensive, portable and sensitive to physiological changes which affect electrical impedance properties. Such imaging may include gastric emptying and ventilation and cardiac output in the thorax. The improved techniques described herein may also be used for EIT imaging of brain function, including imaging acute stroke or epileptic seizures, and may allow portable and low cost systems that have practical advantages over existing methods such as fMRI. For example, these systems may provide images of fast neural activity in the brain over milliseconds.

Multi-frequency EIT (MFEIT) has been shown to be particularly helpful for biological imaging using systems such as cardiac monitoring systems. For example, EIT measurements using the bipolar arrays of electrodes described herein may be used by injection of current at multiple frequencies through an array of skin/scalp bipolar electrodes. 3D impedance distribution maps can be reconstructed by solving the inverse (resistivity/admittivity) problem. As described above, the biological tissue impedance changes with frequency due to the frequency-dependent behavior of cell membranes; each tissue may be characterized by a unique spectroscopic signature. MFEIT, particularly improved as described herein, has the potential to distinguish between hemorrhagic and ischemic brain stroke in emergency situations where CT or MRI are impractical.

The techniques described herein (including the use and implementation of bipolar electrode arrays) may also be used for microscopy, including EIT microscopy. A microscopic electrical impedance tomography (micro-EIT) system may be used for long-term noninvasive monitoring of cell or tissue cultures, and may include a sample container including an array of bipolar electrodes as described herein; any anomaly within the container may perturb the current pathways and therefore equipotential lines to produce different differential voltage data. For example, a modification of the system described in Liu, Q. et al., “Design of a microscopic electrical impedance tomography system using two current injections” (Physiol Meas. 2011 September; 32(9):1505-16) may be made as provided herein.

In general, the methods and apparatuses described herein may be incorporated as part of a neurostimulation electrode array impedance measurement apparatus such as a cochlear implant, spinal cord implant, deep brain implant, peripheral nerve stimulator, transcutaneous electrical nerve stimulation (TENS) device, vagus nerve stimulator and tibial nerve stimulator. In such applications, the number of individual electrode contacts routinely number in the tens or greater, making the technique described herein advantageous.

Any of the techniques described herein may also be used for non-biological applications. For example, the methods and apparatuses for impedance measurements using an array of bipolar electrodes may be particularly useful for geophysics applications. For example, electrical resistivity tomography (ERT) or electrical resistivity imaging (ERI) is a geophysical technique for imaging sub-surface structures from electrical resistivity measurements made at the surface, or by electrodes in one or more boreholes. Electrodes may be suspended in the boreholes to examine deeper sections. The applications of ERT include fault investigation, ground water table investigation, soil moisture content determination and many others. In industrial process imaging ERT can be used in a similar fashion to medical EIT, to image the distribution of conductivity in mixing vessels and pipes. In this context it is usually called Electrical Resistance Tomography.

The methods and techniques described herein may also be applied to architecture, civil engineering and/or archeology. For example, electrical resistivity tomography (ERT), although traditionally used as a surveying tool within archaeology, may also be used as a high-resolution technique that traces the movement of moisture in building materials and provide a vital tool for understanding the decay of buildings including archaeological monuments.

The techniques and apparatuses described above, including the use of bipolar electrode arrays, may also be used for/with microfluidics apparatuses and methods, such as electrophoresis, dielectrophoresis, electrorotation, surface micro fluidics, and the like. In such applications, changes in the observed impedance of a sample under test may be used to inform the status of a test sample (e.g. diagnosis) or the effectivity of a driving force (e.g. pumping). Such systems may routinely employ a multitude of electrode arrays making the technique described herein advantageous.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method of determining electrical properties of a region of a subject's body using bio-impedance of a tissue region, the method including:
attaching a sensor including a plurality of pairs of bipolar electrodes to a skin surface of the subject's body;
applying drive currents at a plurality of different frequencies to bipolar electrode pairs of the plurality of pairs of bipolar electrodes and measuring voltages at the bipolar electrode pairs; and
determining an estimate of electrical properties across at least two of the plurality of different frequencies for a plurality of regions beneath the sensor using the applied drive currents and measured voltages from the plurality of bipolar electrode pairs.

2. The method of claim 1, wherein applying drive currents at the plurality of different frequencies to each of the bipolar electrode pairs includes applying drive currents at a first frequency and at a second frequency.

3. The method of claim 1, wherein attaching the sensor includes attaching a sensor having N electrodes, wherein N is greater than 4.

4. The method of claim 3, wherein attaching the sensor includes attaching a sensor having N electrodes, wherein N is greater than 10.

5. The method of claim 1, wherein determining the estimate includes determining the estimate of electrical properties between a first frequency and a second frequency of the plurality of different frequencies for a plurality of regions beneath the sensor using the applied drive currents and measured voltages from the plurality of bipolar electrode pairs.

6. The method of claim 1, wherein determining the estimate includes determining an estimate of tissue wetness for at least some of the regions of the plurality of regions beneath the sensor.

7. The method of claim 6, wherein the method includes generating an indicator indicative of the estimate of tissue wetness.

8. The method of claim 1, wherein the method includes using a patch sensor including:
a substrate; and,
a plurality of pairs of bipolar electrodes on the substrate, wherein the substrate maintains a predetermined spacing between the electrodes.

9. The method of claim 1, wherein the method includes using an acquisition module to apply drive currents and determine the estimate of electrical properties.

10. The method of claim 1, wherein the method includes generating an indicator indicative of tissue wetness using the using an acquisition module to apply drive currents and determine the estimate of electrical properties.

11. Apparatus for determining electrical properties of a region of a subject's body using bio-impedance of a tissue region, the apparatus including:
a sensor including a plurality of pairs of bipolar electrodes, the sensor being attached to a skin surface of the subject's body in use;
an acquisition module that:
applies drive currents at a plurality of different frequencies to bipolar electrode pairs of the plurality of pairs of bipolar electrodes and measuring voltages at the bipolar electrode pairs; and
determines an estimate of electrical properties across at least two of the plurality of different frequencies for a plurality of regions beneath the sensor using the applied drive currents and measured voltages from the plurality of bipolar electrode pairs.

12. The apparatus of claim 11, wherein the sensor is a patch sensor including:
a substrate; and,
a plurality of pairs of bipolar electrodes on the substrate, wherein the substrate maintains a predetermined spacing between the electrodes.

13. The apparatus of claim 12, wherein the patch sensor includes at least one substrate modification to enhance local flexibility of the substrate so that the patch sensor may conform to a contour of a subject's body,

14. The apparatus of claim 13, wherein the substrate modifications to enhance local flexibility of the substrate include at least one of:
cut-out regions through the substrate;
slits cut through the substrate; and,
regions of material within the substrate having a greater flexibility than the substrate.

15. The apparatus of claim 12, wherein the substrate is flexible and relatively inelastic, so that the spacing between each of the electrodes remains relatively fixed as the sensor is manipulated.

16. The apparatus of claim 12, wherein the patch sensor further includes an adhesive hydrogel.

17. The apparatus of claim 12, wherein the substrate at least one of:
is less than about 5 mils (0.127 mm) thick;
is a polyester material;
is a polyester material and an anti-bacterial titanium oxide material; and,
has a width of between about 0.5 inches (1.3 cm) and about 2.5 inches (6.4 cm).

18. The apparatus of claim 12, wherein the plurality of electrodes include at least one of:
a rectangular shape on the substrate;
silver/silver chloride electrodes;
more than 6 elongate electrodes;
more than 10 electrodes; and,
more than 25 electrodes.

19. The apparatus of claim 11, wherein the acquisition module includes:
an electrode drive unit configured to drive multiple different pairs of electrodes with at least two frequencies; and,
an electrode recording module that allows the acquisition module to record energy from the subject's skin in response to the applied energy between bipolar pairs of electrodes.

20. The apparatus of claim 11, wherein the apparatus includes a data analysis unit that:
receives data from the acquisition unit indicative of the measured voltages and applied drive current;
uses the data to determine the estimate of the electrical properties.

21. (canceled)

22. (canceled)

23. (canceled)

24. (canceled)

25. (canceled)
